(19) United States
(12) Patent Application Publication
(10) Pub. No.: US 2013/0184803 A1
(43) Pub. Date: Jul. 18, 2013

(54) SUBMUCOSAL IMPLANTATION OF NEURAL STIMULATORS

(71) Applicant: Hernan Altman, Haifa (IL)

(72) Inventor: Hernan Altman, Haifa (IL)

(73) Assignee: BRAINSGATE LTD., Caesarea (IL)

(21) Appl. No.: 13/348,731

(22) Filed: Jan. 12, 2012

(51) Int. Cl. A61N 1/05

(52) U.S. Cl. 607/134

(57) ABSTRACT

A method is provided that includes advancing at least one electrode through mucosa of an oral cavity, along a path that does not enter a greater palatine canal, to within 10 mm of a sphenopalatine fossa. The at least one electrode is left implanted within 10 mm of the sphenopalatine fossa. Optionally, the at least one electrode is advanced into the sphenopalatine fossa via a lateral aspect of the fossa, and left implanted in the sphenopalatine fossa. Other embodiments are also described.

### FIELD OF THE APPLICATION

The present invention relates generally to medical procedures and devices. More specifically, the invention relates to medical procedures and devices for stimulating nerve tissue in the head.

### BACKGROUND OF THE APPLICATION

The sphenopalatine ganglion (SPG) (also known as the pterygopalatine ganglion) is a neuronal center located in the sphenopalatine fossa (also known as the pterygopalatine fossa) behind the nose. The SPG consists of parasympathetic neurons innervating the middle cerebral and anterior cerebral lumens, the facial skin blood vessels, and the lacrimal glands. Activation of this ganglion is believed to cause vasodilation of these vessels. A second effect of such stimulation is the opening of pores in the vessel walls, causing plasma protein extravasation (PPE). This effect allows better transport of molecules from within these blood vessels to surrounding tissue.

The greater palatine canal (also known as the pterygopalatine canal) is a passage in the skull that transmits the greater palatine nerve, artery, and vein between the sphenopalatine fossa and the oral cavity. The greater palatine canal is a bony canal that passes through the sphenoid and palatine bones to reach the palate via the greater palatine foramen.

### SUMMARY OF THE INVENTION

Some applications of the present invention provide surgical techniques for implanting a neural stimulator such that at least one electrode thereof is positioned in or in a vicinity of the sphenopalatine fossa in a vicinity of the sphenopalatine ganglion (SPG). The sphenopalatine fossa is accessed from its lateral aspect, where the fossa is open (i.e., has no lateral bony wall) and separated from its surrounding only by soft tissue.

For some applications, the neural stimulator is advanced toward the sphenopalatine fossa along a submucosal path that:
begins in the oral cavity;
passes through mucosa at a lateral aspect of the alveolar process, in a vicinity of the junction between the maxilla and the pterygoid process of the sphenoid bone;
progresses superiorly (towards the top of the patient's skull) through soft tissue for between 10 and 20 mm; and
passes through the lateral aspect of the sphenopalatine fossa into the fossa.

The path may be curved medially and/or antero-posteriorly. This curvature may be followed either by mechanically “hugging”/pressing towards the alveolar process of the maxillary bone and/or by navigation using imaging, such as computerized tomography (CT) or magnetic resonance imaging (MRI).

This approach to the fossa thus bypasses the greater palatine canal, thereby avoiding the challenges associated with passage through the canal, including surgical preparation of the fragile bony canal. The path progresses superiorly, at least one point on the path being between 1 and 10 mm posterolateral to the canal, passing generally alongside greater the palatine canal.

In experiments conducted by the inventor, the technical feasibility of this submucosal surgical approach has been demonstrated in skulls and in human cadavers.

Once implanted, the stimulator typically delivers energy to the SPG or another parasympathetic site (such as those described in the patent applications incorporated hereinbelow by reference) in order to activate the SPG or other site, to control and/or modify SPG-related behavior, e.g., in order to induce changes in cerebral blood flow and/or to modulate permeability of the blood-brain-barrier (BBB).

The stimulation may be used in many medical applications, such as, by way of illustration and not limitation:
the treatment or prevention of cerebrovascular disorders such as stroke or other adverse brain events, such as described in U.S. application Ser. No. 11/465,381, which issued as U.S. Pat. No. 8,055,347;
the enhancement of neurogenesis or brain metabolic activity, such as described in U.S. application Ser. No. 12/197,614, which published as US Patent Application Publication 2009/0210026;
the treatment or prevention of vascular dementia (VaD), such as described in U.S. application Ser. No. 11/874,529, which issued as U.S. Pat. No. 7,860,569;
the treatment or prevention of Alzheimer's disease, such as described in U.S. application Ser. No. 10/518,322, which issued as U.S. Pat. No. 7,640,062, and/or U.S. application Ser. No. 11/874,529, which issued as U.S. Pat. No. 7,860,569;
the facilitation of drug transport across the BBB, such as described in U.S. Pat. No. 7,120,489.

Alternatively, the stimulator is configured to inhibit the SPG or other site, such as in order to treat migraine headaches.

There is therefore provided, in accordance with an application of the present invention, a method including:

advancing at least one electrode through mucosa of an oral cavity, along a path that does not enter a greater palatine canal, to within 10 mm of a sphenopalatine fossa; and

leaving the at least one electrode implanted within 10 mm of the sphenopalatine fossa.

For some applications, advancing includes advancing the at least one electrode to within 3 mm of the sphenopalatine fossa, and leaving includes leaving the at least one electrode implanted within 3 mm of the sphenopalatine fossa.

For some applications, advancing including advancing the at least one electrode into the sphenopalatine fossa via a lateral aspect of the fossa, and leaving includes leaving the at least one electrode implanted in the sphenopalatine fossa.

Optionally, advancing includes advancing the at least one electrode superiorly such that at least one point of the path is between 1 and 10 mm posterolateral to the greater palatine canal.

For some applications, the path: (a) passes through the mucosa at a lateral aspect of an alveolar process of a maxilla, in a vicinity of a junction between the maxilla and a pterygoid process of a sphenoid bone, (b) subsequently progresses superiorly through soft tissue, and (c) subsequently passes to within 10 mm of the sphenopalatine fossa, and advancing includes advancing the at least one electrode along the path. For some applications, the path passes through the lateral aspect of the sphenopalatine fossa into the sphenopalatine fossa, and advancing includes advancing the at least one electrode along the path.

For any of the applications described above, advancing may include advancing the at least one electrode superiorly such that at least one point of the path is between 1 and 10 mm posterolateral to the greater palatine canal.

For any of the applications described above, advancing may include advancing the at least one electrode superiorly such that at least one point of the path is between 1 and 10 mm posterolateral to the greater palatine canal.

For any of the applications described above, the path may be curved in a direction selected from the group of directions consisting of: medially, antero-posteriorly, and both medially and antero-posteriorly, and advancing may include advancing the at least one electrode along the curved path.

For any of the applications described above, advancing may include mechanically pressing the at least one electrode towards a junction of an alveolar process and a sphenoid bone while advancing.

For any of the applications described above, advancing may include advancing the at least one electrode through the mucosa at a point of incision through the mucosa that is located at a height of a mucobuccal fold between second and third molars.

For any of the applications described above, the method may further include applying an electrical current, using the at least one electrode, to at least one site selected from the group consisting of: an SPG, and another parasympathetic site in a vicinity of the sphenopalatine fossa.

The present invention will be more fully understood from the following detailed description of the embodiments thereof, taken together with the drawings in which:

### BRIEF DESCRIPTION OF THE DRAWINGS

FIG. 1 is a schematic illustration of a surgical technique for implanting a neural stimulator, in accordance with an application of the present invention; and

FIG. 2 is a schematic illustration of an introducer tool and a neural stimulator, in accordance with an application of the present invention.

### DETAILED DESCRIPTION OF APPLICATIONS

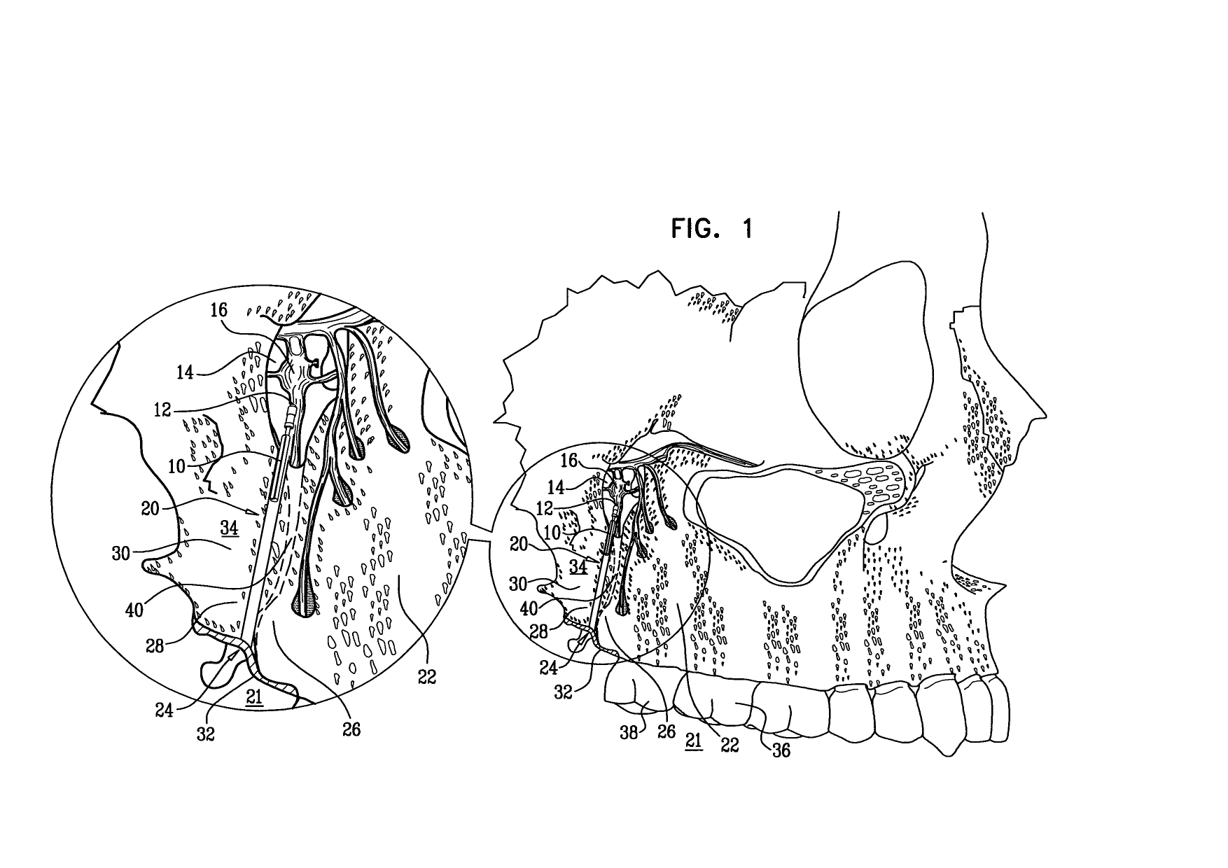

FIG. 1 is a schematic illustration of a surgical technique for implanting a neural stimulator 10, in accordance with an application of the present invention. In this surgical technique, neural stimulator 10 is implanted such that at least one electrode 12 thereof is positioned in or in a vicinity of (e.g., within 10 mm, such as within 3 mm of) a sphenopalatine fossa 14 in a vicinity of a sphenopalatine ganglion (SPG) 16. The sphenopalatine fossa is accessed from its lateral aspect, where the fossa is open (i.e., has no lateral bony wall) and separated from its surrounding only by soft tissue. The length of neural stimulator 10 is shown by way of example in FIG. 1, and may be longer or shorter.

For some applications, at least one electrode 12 of neural stimulator 10 is advanced toward sphenopalatine fossa 14 along a submucosal path 20 that:
begins in an oral cavity 21;
passes through mucosa 32 at a lateral aspect of an alveolar process 22 of a maxilla 26, in a vicinity of a junction 24 between maxilla 26 and a pterygoid process 28 of a sphenoid bone 30 (hereinbelow, the “spheno-maxillary junction” (“SMJ”) 24);
subsequently progresses superiorly (towards the top of the patient's skull) through soft tissue 34 for between 10 and 20 mm, to alveolar process 22 of maxilla 26; and
subsequently passes through the lateral opening of sphenopalatine fossa 14, into the fossa or to a vicinity of sphenopalatine fossa 14 (e.g., within 10 mm, such as within 3 mm of sphenopalatine fossa 14).

After being thus advanced, at least one electrode 12 is left in place, implanted in or in the vicinity of sphenopalatine fossa 14, e.g., within 10 mm, such as within 3 mm, of the fossa. Neural stimulator 10, e.g., at least one electrode 12 thereof, may or may not comprise one or more elements for holding at least one electrode 12 in place in the tissue.

The site of the incision through mucosa 32 may be located using the anatomical landmarks of second and third upper molars 36 and 38. For some applications, such as when performing a “blind” procedure (without radiological guidance), the insertion point is at the height of the mucobuccal fold between the second and third molars 36 and 38. The path of insertion is generally directed superiorly, medially, and posteriorly from this insertion point. Alternatively, for applications in which the procedure is performed with radiological navigation, the path may be planned upfront on a radiological image (e.g., a CT image), in which case the path need not be defined based on anatomy. For some applications, a surgical punch is used to make the incision in the mucosa, while for other applications, a distal end of neural stimulator 10 or an introducer tool is shaped so as to define a sharp tip that serves as a punch.

Path 20 may be curved medially and/or antero-posteriorly. This curvature may be followed either by mechanically “hugging”/pressing towards a junction of alveolar process 22 and sphenoid bone 30, and/or by navigation using imaging, such as computerized tomography (CT) or magnetic resonance imaging (MRI).

This approach to fossa 14 thus bypasses a greater palatine canal 40, thereby avoiding the challenges associated with passage through the canal, including, in some cases, surgical preparation of the fragile bony canal. Path 20 progresses superiorly, at least one point on the path being between 1 and 10 mm posterolateral to the canal, generally passing alongside greater palatine canal 40.

For some applications, in which neural stimulator comprises two or more electrodes 12, fewer than all of the electrodes are positioned in sphenopalatine fossa 14 at the completion of the implantation procedure. The remaining one or more electrodes are positioned outside the fossa in a vicinity thereof. For example, a single distal-most electrode may be positioned within the fossa, while one or more proximal electrodes may be positioned outside the fossa in a vicinity thereof.

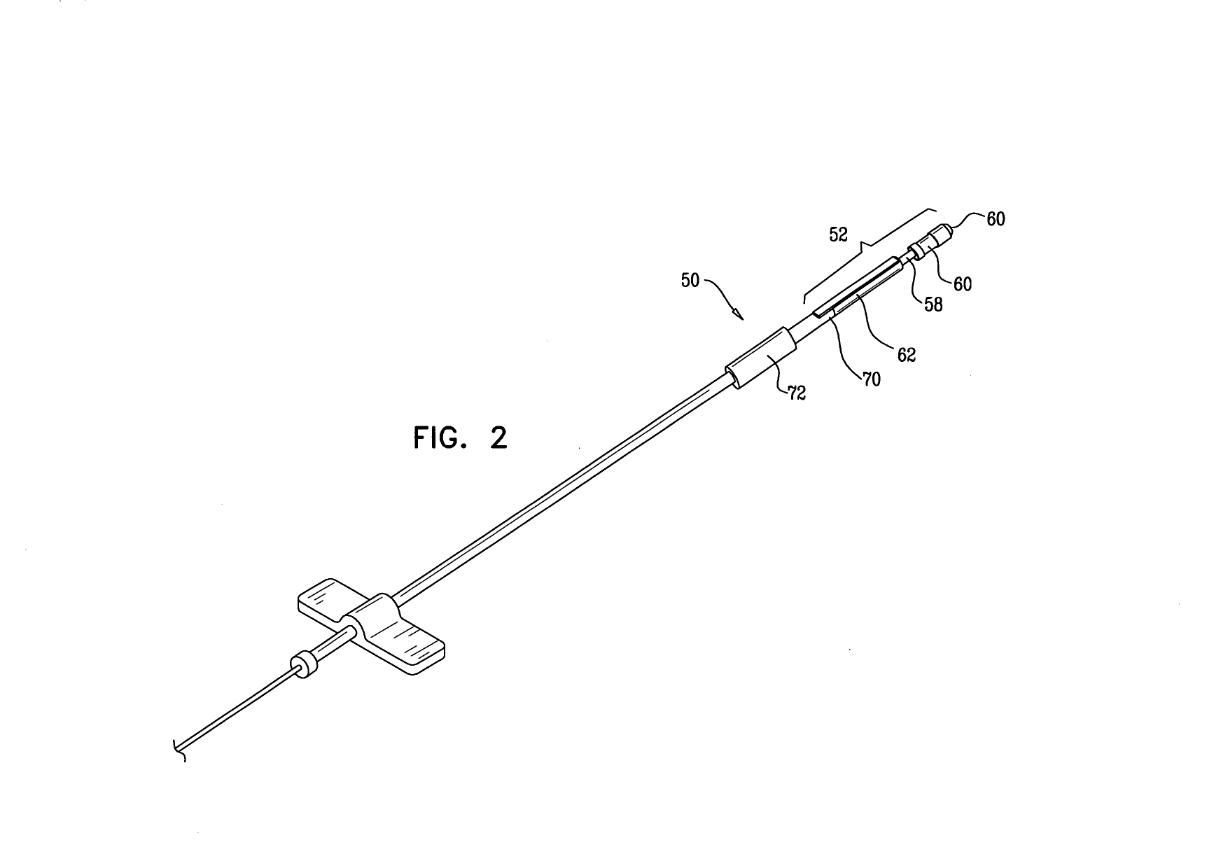

For some applications, neural stimulator 10 is advanced using an introducer tool, such as introducer tool 50 described hereinbelow with reference to FIG. 2. Alternatively, other means are used to convey the stimulator through the soft tissue to its target. Optionally, neural stimulator 10 comprises an elongated support element that remains in at least a portion of the path of insertion.

Once implanted, neural stimulator 10 typically delivers energy to SPG 16 or another parasympathetic site (such as those described in the patent applications incorporated hereinbelow by reference, e.g., a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, and/or or a peripheral site that is directly or indirectly efferently innervated by the SPG), in order to activate the SPG or other site, to control and/or modify SPG-related behavior, e.g., in order to induce changes in cerebral blood flow and/or to modulate permeability of the blood-brain-barrier (BBB).

The stimulation may be used in many medical applications, such as, by way of illustration and not limitation:
the treatment or prevention of cerebrovascular disorders such as stroke or other adverse brain events, such as described in U.S. application Ser. No. 11/465,381, which issued as U.S. Pat. No. 8,055,347;
the enhancement of neurogenesis or brain metabolic activity, such as described in U.S. application Ser. No. 12/197,614, which published as US Patent Application Publication 2009/0210026;
the treatment or prevention of vascular dementia (VaD), such as described in U.S. application Ser. No. 11/874,529, which issued as U.S. Pat. No. 7,860,569;
the treatment or prevention of Alzheimer's disease, such as described in U.S. application Ser. No. 10/518,322, which issued as U.S. Pat. No. 7,640,062, and/or U.S. application Ser. No. 11/874,529, which issued as U.S. Pat. No. 7,860,569;
the facilitation of drug transport across the BBB, such as described in U.S. Pat. No. 7,120,489.

Alternatively, the stimulator is configured to inhibit the SPG or other site, such as in order to treat migraine headaches.

Reference is now made to FIG. 2, which is a schematic illustration of an introducer tool 50 and a neural stimulator 52, in accordance with an application of the present invention. Neural stimulator 10, described hereinabove with reference to FIG. 1, may comprise neural stimulator 52, or another neural stimulator.

Neural stimulator 52 typically comprises an elongated support element 58, one or more electrodes 60 fixed to the support element in a vicinity of a distal end thereof, and circuitry 62 coupled to the support element in a vicinity of a proximal end thereof. The outer surface of the support element is typically electrically insulated along the length thereof. Neural stimulator 52 may incorporate apparatus and techniques described in one or more of the patent applications incorporated by reference hereinbelow.

A distal end of introducer tool 50 comprises a coupling element 70, to which a proximal end of neural stimulator 52 is temporarily coupled prior to performing the implantation procedure. For example, a cord may be coupled to the proximal end of the neural stimulator, pass through the introducer tool, and be temporarily coupled to the proximal end of the introducer tool (e.g., with a knot) such that the cord is tense and holds the neural stimulator tightly to coupling element 70 (cord not shown in FIG. 2). For example, the cord may comprise a suture, string, or wire.

For some applications, introducer tool 50 comprises a collar 72, which is configured to limit a depth of insertion of the introducer tool into path 20. For example, the collar may be configured to limit the depth of insertion of the distal tip of the neural stimulator to the estimated distance from SMJ 24 to the SPG in a typical patient, e.g., between about 10 and about 35 mm. For some applications, the collar comprises a plastic tube placed around all or a portion of the shaft of the introducer tool.

During the implantation procedure described hereinabove with reference to FIG. 1, when the introducer and neural stimulator have reached the desired implantation location, the neural stimulator is decoupled from the introducer tool (such as by cutting the cord). The introducer tool is withdrawn, leaving the distal end of the neural stimulator implanted in the vicinity of the SPG, and at least a proximal portion of support element 58 implanted in path 20 surrounded by soft tissue 34.

For some applications, verification and/or optimization of suitable interfacing of the electrodes with tissue after the electrodes are placed is performed by observing the effects of stimulation on one or more physiological responses. Such observations include, but are not limited to: (1) evaluating the vasodilatation of blood vessels of the eye, (2) assessment of cerebral blood flow (e.g., changes in blood flow) by using Doppler (e.g., transcranial Doppler or laser Doppler), (3) assessment of forehead perfusion by using Laser-Doppler, and (4) assessment of forehead perfusion by a temperature sensor.

For some applications, techniques described herein are practiced in combination with techniques described in US Patent Application Publication 2006/0195169 to Gross et al., entitled, “Surgical tools and techniques for stimulation,” which is incorporated herein by reference.

Techniques described in this application may be practiced in combination with methods and apparatus described in one or more of the following patent applications, which are assigned to the assignee of the present patent application and are incorporated herein by reference:
U.S. Provisional Patent Application 60/203,172, filed May 8, 2000, entitled, “Method and apparatus for stimulating the sphenopalatine ganglion to modify properties of the BBB and cerebral blood flow”
U.S. patent application Ser. No. 10/258,714, filed Oct. 25, 2002, entitled, “Method and apparatus for stimulating the sphenopalatine ganglion to modify properties of the BBB and cerebral blood flow,” which issued as U.S. Pat. No. 7,120,489, or the above-referenced PCT Publication WO 01/85094
U.S. Provisional Patent Application 60/364,451, filed Mar. 15, 2002, entitled, “Applications of stimulating the sphenopalatine ganglion (SPG)”
U.S. Provisional Patent Application 60/368,657, filed Mar. 28, 2002, entitled, “SPG Stimulation”
U.S. Provisional Patent Application 60/376,048, filed Apr. 25, 2002, entitled, “Methods and apparatus for modifying properties of the BBB and cerebral circulation by using the neuroexcitatory and/or neuroinhibitory effects of odorants on nerves in the head”
U.S. Provisional Patent Application 60/388,931, filed Jun. 14, 2002, entitled “Methods and systems for management of Alzheimer's disease,” PCT Patent Application PCT/IL03/000508, filed Jun. 13, 2003, claiming priority therefrom, and U.S. Pat. No. 7,640,062 in the national stage thereof
U.S. Provisional Patent Application 60/400,167, filed Jul. 31, 2002, entitled, “Delivering compounds to the brain by modifying properties of the BBB and cerebral circulation”
U.S. Provisional Patent Application 60/426,180, filed Nov. 14, 2002, entitled, “Surgical tools and techniques for sphenopalatine ganglion stimulation,” PCT Patent Application PCT/IL03/000966, filed Nov. 13, 2003, which claims priority therefrom, and U.S. Pat. No. 7,636,597 in the national stage thereof
U.S. Provisional Patent Application 60/426,182, filed Nov. 14, 2002, and corresponding PCT Patent Application PCT/IL03/000967, which claims priority therefrom, filed Nov. 13, 2003, entitled, “Stimulation circuitry and control of electronic medical device,” which published as PCT Publication WO 04/044947, and a U.S. patent application filed May 11, 2005 in the national stage thereof
U.S. patent application Ser. No. 10/294,310, filed Nov. 14, 2002, entitled, “SPG stimulation for treating eye pathologies,” which published as US Patent Application Publication 2003/0176898, and PCT Patent Application PCT/IL03/000965, filed Nov. 13, 2003, claiming priority therefrom
PCT Patent Application PCT/IL03/000631, filed Jul. 31, 2003, entitled, “Delivering compounds to the brain by modifying properties of the BBB and cerebral circulation,” which published as PCT Publication WO 04/010923, and U.S. patent application Ser. No. 10/522,615 in the national stage thereof
U.S. Pat. No. 6,853,858 to Shalev
U.S. patent application Ser. No. 10/783,113, filed Feb. 20, 2004, entitled, “Stimulation for acute conditions,” which published as US Patent Application Publication 2004/0220644
U.S. Provisional Patent Application 60/426,181, filed Nov. 14, 2002, entitled, “Stimulation for treating ear pathologies,” PCT Patent Application PCT/IL03/000963, filed Nov. 13, 2003, which claims priority therefrom, and which published as PCT Publication WO 04/045242, and U.S. patent application Ser. No. 10/535,025 in the national stage thereof
U.S. Provisional Patent Application 60/448,807, filed Feb. 20, 2003, entitled, “Stimulation for treating autoimmune-related disorders of the CNS”
U.S. Provisional Patent Application 60/461,232 to Gross et al., filed Apr. 8, 2003, entitled, “Treating abnormal conditions of the mind and body by modifying properties of the blood-brain barrier and cephalic blood flow”
PCT Patent Application PCT/IL03/00338 to Shalev, filed Apr. 25, 2003, entitled, “Methods and apparatus for modifying properties of the BBB and cerebral circulation by using the neuroexcitatory and/or neuroinhibitory effects of odorants on nerves in the head,” and U.S. patent application Ser. No. 10/512,780, filed Oct. 25, 2004 in the national stage thereof, which published as US Patent Application Publication 2005/0266099
U.S. Provisional Patent Application 60/506,165, filed Sep. 26, 2003, entitled, “Diagnostic applications of stimulation”
U.S. patent application Ser. No. 10/678,730, filed Oct. 2, 2003, entitled, “Targeted release of nitric oxide in the brain circulation for opening the BBB,” which published as US Patent Application Publication 2005/0074506, and PCT Patent Application PCT/IL04/000911, filed Oct. 3, 2004, claiming priority therefrom
PCT Patent Application PCT/IL04/000897, filed Sep. 26, 2004, entitled, “Stimulation for treating and diagnosing conditions,” which published as PCT Publication WO 05/030025
U.S. Provisional Patent Application 60/604,037, filed Aug. 23, 2004, entitled, “Concurrent bilateral SPG modulation”
PCT Patent Application PCT/IL05/000912, filed Aug. 23, 2005, entitled, “Concurrent bilateral SPG modulation,” which published as PCT Publication WO 06/021957
U.S. patent application Ser. No. 10/952,536, filed Sep. 27, 2004, entitled, “Stimulation for treating and diagnosing conditions,” which published as US Patent Application Publication 2005/0159790
U.S. patent application Ser. No. 11/349,020, filed Feb. 7, 2006, entitled, “SPG stimulation via the greater palatine canal,” which issued as U.S. Pat. No. 7,561,919
U.S. patent application Ser. No. 11/465,381, filed Aug. 17, 2006, entitled, “Stimulation for treating brain events and other conditions,” which published as US Patent Application Publication 2007/0083245
U.S. patent application Ser. No. 11/668,305, filed Jan. 19, 2007, entitled, “Stimulation of the otic ganglion for treating medical conditions,” which issued as U.S. Pat. No. 7,684,859
U.S. Provisional Application 61/195,556, filed Oct. 7, 2008, entitled, “Detection of electrode position for SPG stimulation by measurement of electrical effects”
U.S. patent application Ser. No. 12/197,614, filed Aug. 25, 2008, entitled, “SPG stimulation for enhancing neurogenesis and brain metabolism,” which published as US Patent Application Publication 2009/0210026
U.S. patent application Ser. No. 11/874,529, filed Oct. 18, 2007, entitled, “Long-term SPG stimulation therapy for prevention of vascular dementia,” which published as US Patent Application Publication 2009/0105783
U.S. patent application Ser. No. 12/575,165, filed Oct. 7, 2009, entitled, “Flexible tools for preparing bony canals,” which published as US Patent Application Publication 2010/0114184

It is noted that the figures depicting applications of the present invention are not necessarily drawn to scale, and, instead, may change certain dimensions in order to more clearly demonstrate some aspects of the invention.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
advancing at least one electrode through mucosa of an oral cavity, along a path that does not enter a greater palatine canal, to within 10 mm of a sphenopalatine fossa; and
leaving the at least one electrode implanted within 10 mm of the sphenopalatine fossa.

2. The method according to claim 1, wherein advancing comprises advancing the at least one electrode to within 3 mm of the sphenopalatine fossa, and wherein leaving comprises leaving the at least one electrode implanted within 3 mm of the sphenopalatine fossa.

3. The method according to claim 1, wherein advancing comprising advancing the at least one electrode into the sphenopalatine fossa via a lateral aspect of the fossa, and wherein leaving comprises leaving the at least one electrode implanted in the sphenopalatine fossa.

4. The method according to claim 3, wherein advancing comprises advancing the at least one electrode superiorly such that at least one point of the path is between 1 and 10 mm posterolateral to the greater palatine canal.

5. The method according to claim 3, wherein the path is curved in a direction selected from the group of directions consisting of: medially, antero-posteriorly, and both medially and antero-posteriorly, and wherein advancing comprises advancing the at least one electrode along the curved path.

6. The method according to claim 3, wherein advancing comprises mechanically pressing the at least one electrode towards a junction of an alveolar process and a sphenoid bone while advancing.

7. The method according to claim 3, wherein advancing comprises advancing the at least one electrode through the mucosa at a point of incision through the mucosa that is located at a height of a mucobuccal fold between second and third molars.

8. The method according to claim 3, further comprising applying an electrical current, using the at least one electrode, to at least one site selected from the group consisting of: an SPG, and another parasympathetic site in a vicinity of the sphenopalatine fossa.

9. The method according to claim 1, wherein the path:
passes through the mucosa at a lateral aspect of an alveolar process of a maxilla, in a vicinity of a junction between the maxilla and a pterygoid process of a sphenoid bone,
subsequently progresses superiorly through soft tissue, and
subsequently passes to within 10 mm of the sphenopalatine fossa, and
wherein advancing comprises advancing the at least one electrode along the path.

10. The method according to claim 9, wherein the path passes through the lateral aspect of the sphenopalatine fossa into the sphenopalatine fossa, and wherein advancing comprises advancing the at least one electrode along the path.

11. The method according to claim 9, wherein advancing comprises advancing the at least one electrode superiorly such that at least one point of the path is between 1 and 10 mm posterolateral to the greater palatine canal.

12. The method according to claim 1, wherein advancing comprises advancing the at least one electrode superiorly such that at least one point of the path is between 1 and 10 mm posterolateral to the greater palatine canal.

13. The method according to claim 1, wherein the path is curved in a direction selected from the group of directions consisting of: medially, antero-posteriorly, and both medially and antero-posteriorly, and wherein advancing comprises advancing the at least one electrode along the curved path.

14. The method according to claim 1, wherein advancing comprises mechanically pressing the at least one electrode towards a junction of an alveolar process and a sphenoid bone while advancing.

15. The method according to claim 1, wherein advancing comprises advancing the at least one electrode through the mucosa at a point of incision through the mucosa that is located at a height of a mucobuccal fold between second and third molars.

16. The method according to claim 1, further comprising applying an electrical current, using the at least one electrode, to at least one site selected from the group consisting of: an SPG, and another parasympathetic site in a vicinity of the sphenopalatine fossa.
